(19) United States
(12) Patent Application Publication
(10) Pub. No.: US 2010/0298925 A1
(43) Pub. Date: Nov. 25, 2010

(54) SPINULOSE METAL SURFACES

(75) Inventors: CHRISTINA K. THOMAS, ST. PAUL, MN (US); DANIEL M. STOREY, MINNEAPOLIS, MN (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: CHAMELEON SCIENTIFIC CORPORATION, PLYMOUTH, MN (US)

(21) Appl. No.: 12/816,827

(22) Filed: Jun. 16, 2010

Related U.S. Application Data
(63) Continuation-in-part of application No. 12/152,698, filed on May 16, 2008, now abandoned.
Application No. 12/152,698 is a continuation-in-part of application No. 11/932,831, filed on Oct. 31, 2007.

(51) Int. Cl. A61F 2/06; B32B 3/10; C12N 11/08

(52) U.S. Cl. 623/1.15; 428/148; 428/144; 435/180

(57) ABSTRACT

Spinulose metal surfaces are produced by a modified nanoplasma cyclic deposition process. The unique spinulose surfaces are highly adherent toward polymer and bioactive molecules and cells, including osteoblast, fibroblast and endothelial cells. The nanostructured spinulose surfaces can be coated with a wide range of polymers to form polymer surface coatings that are particularly useful on implants, catheters, guidewires, stents and other medical devices intended for in vivo applications.

### CROSS-REFERENCE TO RELATED APPLICATIONS

This application is a continuation-in-part of U.S. Ser. No. 12/152,698, filed May 16, 2008, which is a continuation-in-part of U.S. Ser. No. 11/932,831, filed Oct. 31, 2007, the disclosures of which are hereby incorporated by reference in their entirety, including all figures, tables and amino acid or nucleic acid sequences.

### BACKGROUND OF THE INVENTION

1. Field of the Invention

The invention relates to structured surfaces and films, and particularly to nanostructured spinulose surfaces produced by modified plasma vapor deposition of a vaporizable material onto a surface.

2. Description of Background Art

Surface films and surface modifications are increasingly important in the development of biocompatible surfaces for medical devices and for protective coverings on materials susceptible to external damage. A particular area of interest is the engineering of surfaces that act as scaffolds for cell adhesion or have immobilization properties that can be modified for time release of bioactive agents such as drugs.

Several techniques, including physical vapor deposition, for producing nanostructured thin films have been studied over the past several years. Metal oxides deposited at glancing angles result in controllable columnar microstructures depending on substrate motion variation (Robbie and Brett, 1997). The glancing angle technique (GLAD), produces vapor deposited thin film microstructures with a distinct helical columnar appearance (U.S. Pat. No. 6,248,422; U.S. Pat. No. 6,206,065).

Nanostructured surfaces of GLAD films have been suggested as having possible applications in chiral optics and, due to magnetic anisotropy, possible development of information storage devices because of the ability to deposit materials such as silicon in the form of nanostructured helical columns. Hawkeye and Brett (2007) reviewed GLAD films and foresee applications in solar energy conversion, fuel cells, gas sensors, catalysts and electrochemical capacitors.

Films produced from electron beam heated silicon deposited on glass were studied by McIntosh, et al. (2003) to assess hTert fibroblast morphology and survival on the columnar surfaces created from silicon deposited over a range of angles. Adhesion, spreading and survival beyond one day were observed only on surfaces deposited at a 70° angle, despite the identical composition of the columns deposited at other angles.

Corrosion is a persistent problem with metals exposed to air and water; for example, the harsh environments encountered by steel rebars used in highways and bridges has led to increased use of deicing salts, which has accelerated corrosion damage. Various films on metal surfaces have been investigated in efforts to develop suitable protective coatings.

Polymer coatings on metals are found to be useful in several applications, ranging from corrosion-inhibiting surfaces to biocompatible thin films on medical devices. Polymers with low coefficients of friction are desirable in catheters and guidewires used in surgical procedures and in permanently implanted devices such as stents and valves. Metals are used in the fabrication of several types of implants; however, bare metals used in stents, for example, may provide a focus for restenosis, due to neointimal proliferation subsequent to implantation. Polymer coated stents have, in some instances, appeared to reduce the potential for the inflammation and thrombogenic reactions leading to restenosis. Many polymers are not suitable for implanted devices because of flexing or expansion upon implantation, in addition to peeling, cracking or detachment from the underlying metal substrate.

Several different types of polymers have been described as having properties useful for medical device coatings, ranging from polymers covalently attached to a metal surface to thin hydrogel films and biodegradable coatings.

Biocompatibility of the coating polymers used on implant devices is important. Billinger, et al. ((2006) reported decreased inflammation from poly(L-lysine)-graft-(polyethylene)glycol (PLL-g-PEG) coating, which appears to reduce cell-stent interactions.

WO/1995/004839 describes pretreating metal guidewires with a hydrocarbon plasma deposited residue over the metal, which in turn acts as a tie layer for a subsequently applied outer hydrophilic polymer coating.

Other “layering” techniques have been used to prepare polymer-coated metal surfaces. U.S. Pat. No. 6,235,361 describes a metal surface coated with a thermoplastic polymer which has a peel strength at 130° C. An epoxy resin and a polypropylene binder are placed between the metal surface and a thermoplastic layer.

Polymer films have been textured to provide enhanced adhesion of plasma deposited metals. The morphology of the polymer surface is characterized by mounds and dimples, but the adherence of the polymer to an underlying surface is not addressed and the polymer structured surface is dependent on regulation of polymer phase kinetics (U.S. Pat. No. 6,099,939).

Many polymer coatings are not satisfactory for all types of surfaces, particularly for metal surfaces where a coating could provide protection from oxidative processes or increase or add desirable properties such as lubricity. The sloughing and peeling encountered with some polymer coated metal surfaces shows a lack of strong surface adherence to the substrate. This is of particular concern and interest in the development of biocompatible coatings on medical implants and other medical devices because the biocompatible properties of certain classes of polymers make them otherwise ideal for use on implants and other types of devices used in vivo.

### EMBODIMENTS OF THE INVENTION

Methods for producing metal spinulose surfaces using a modified plasma vapor deposition are disclosed. The present invention solves many of the troublesome problems frequently encountered with sloughing and peeling of polymers used to coat and protect surfaces, particularly the biocompatible polymers currently used to coat surfaces of medical devices. Spinulose metal surfaces and films promote strong cell adherence and when coated with a polymer will retain the spinulose nanostructural features that promote cell adherence.

The spinulose surfaces of the present invention are unique and distinctly different in physical appearance from the metal “whiskers” often seen in digital circuits as tiny hair-like projections. The surfaces and films produced by the disclosed vapor deposition methods have a spiney or spike-like appearance with pointy projections over the surface, which are readily distinguished from columnar or rod-like structures and are distinctly different from other reported nanostructured surfaces. The burr-like nanoparticulates seen under the SEM on the surface range in size from about 1 to about 10 μm in diameter. The pointy projections have a slight wedge shape and are relatively short, varying in length from about 0.01 μm to 0.1 μm. The appearance is like the prickly envelope of a fruit.

Physical vapor deposition (PVD) is used to describe a class of processes that involve the deposition of material, often in the form of a thin film, from a condensable vapor which has been produced from a solid precursor by physical means. There are many ways of producing the vapor, and many modifications to each of these processes. Examples of PVD processes include evaporation, sputtering, laser ablation and arc discharge. PVD can involve chemical reactions, such as from multiple sources, or by addition of a reactive gas.

The nano plasma deposition (NPD) method to produce spinulose surfaces is a modified form of physical vapor deposition (PVD). Several features of the method are distinguishable from currently used methods for forming nanostructured metal surfaces by physical vapor deposition. The majority of reported deposition methods, as discussed, are vapor-liquid-solid (or vapor-solid), chemical vapor deposition (CVD) processes or electron beam evaporation. The present invention utilizes a process based on vapor deposition, employing a plasma arc deposition procedure where low voltage, (<100 V), high current, (>5 A), discharge ablates a metal cathode in an evacuated chamber and an inert atmosphere so that the metal is deposited onto a substrate surface. Unexpectedly, unique spike-like nanostructural surface features are obtained when, contrary to conventionally used procedures, metal vapor deposition is periodically cycled by reducing inert gas flow and plasma discharge for selected intervals, as set forth in detail in the examples.

The novel spinulose surface of a nano plasma deposited (NPD) metal using the described conditions exhibits features significantly different in appearance from previously reported vapor deposited metals and metal compounds. As cycling of the metal plasma progresses, nano-roughness appears during the deposition process as spikes on round particulates. The spikes can be controlled in height and number by the number of cycles employed, which is a relatively small number on the order of about 3 up to at least 15 or so, at least for the particular metal examples used to illustrate the invention. Spinulose surfaces are believed to be possible with a range of metals, although it is expected that some modifications may be required in the cycling conditions.

While the invention is illustrated with deposition of titanium (Ti) and zirconium (Zr) spinulose coatings on several commonly used substrates, the novel spinulose surfaces can be created as films without the supporting substrate. Ti or Zr, for example, may be deposited on a carbon substrate and the resulting spinulose film, can be isolated by burning off the carbon. Other readily removable or degradable substrates can be envisioned, such as those which can be easily removed without altering the integrity of the film by dissolving a salt or similar dissolvable substrate.

Substrates suitable as temporary matrices for film deposition include various salts. Sodium or potassium chloride, for example, can be readily dissolved after NPD deposition of Ti or other metals. The particulate surface remaining after dissolution can be recovered as a film or powder and used as a high surface area catalyst in bioreactors or in a number of other applications based on the unique nanostructure. As drug delivery vehicles, spinulose and other nanostructured particles with low surface energy can act as a reservoir or support for chemicals or biomolecules. A slowly dissolving salt matrix for example can be used to release an attached drug in a time dependent manner.

The nanostruetural features of metals deposited by the described NPD method are different from ion plasma deposited films where deposition is conducted for specified time periods at different voltages or by varying the other deposition parameters. Serendipitously, it was found that a cycling or intermittent deposition from metal targets produced the unexpected spinulose surface features on the deposited metal films. While globular nanostructures were observed with cobalt, copper, nickel, hafnium, 316L stainless steel, nitanol, titanium 6-4, and silver, titanium and zirconium formed distinct spinulose nanostructured surfaces. Aluminum exhibited distinct surface features under the deposition conditions, although the surface was devoid of spikes and globules and appeared as bead-like rounded structures interspersed between larger round particles.

Aluminum metal deposited under the same conditions described for Ti and Zr has a stacked appearance with a geometric cube-like structure different from the structures observed with Ti and other metals. While spinulose surfaces for aluminum and other metals were not observed under the conditions used to produce spinulose Ti nanostructured surfaces, it may be possible to generate spinules by using modifications of the disclosed deposition procedures, such as, but not necessarily limited to, longer intervals between deposition cycles, distance from target and chamber pressure.

Spinulose nanostructured Ti surfaces can be formed as coatings or films on virtually any metal, plastic or ceramic surface, including stainless steel, titanium, CoCrMo, nitinol, glass or silicon, as well as on silicone, poly(methylmethacrylate) (PMMA), polyurethane (PU), polyvinyl chloride (PVC), polyethylene terephthalate glycol (PETG), polyetheretherketone (PEEK), polytetrafluoroethylene (PTFE), polyethylene terephthalate (PET), ultra high molecular weight polyethylene (UHMWPE), and polypropylene (PP). Other metals, including aluminum, gold, platinum and silver are also suitable substrates.

A particular embodiment of the invention is a polymer-coated titanium or zirconium spinulose surface. Titanium or zirconium spinulose surfaces or films can be prepared on any type of substrate whether metal, polymer, glass, or ceramic The spinulose nanostructured substrate surfaces produced by the modified NPD method demonstrates that under certain controlled deposition conditions, a unique “spikey” metal film or coating can be produced on virtually any substrate. The present invention demonstrates that such spikey surfaces generated from titanium or zirconium are surprisingly well suited for top coating with a wide range of polymers. Appropriate polymers can be selected as required for specialized utilities such as protective coatings, anchors or matrices, biocompatibility and controlled elution coatings.

Using the procedures described herein, polymers are durably attached to surfaces that would otherwise exhibit only weak or unpredictable attachment polymer attachment. The thickness of polymer films can be controlled by the deposition method; for example, several dipping steps after initial dipping or formation of a polymer layer on a spinulose surface can be used to provide thicknesses varying up to several microns.

The unique structure of the spinulose surface is produced by controlled nanoplasma deposition. A polymer can be dispersed on this surface also using a vapor deposition method, but in some cases more conveniently by simple dipping. Many agents, including bioactive materials such as therapeutic drugs, can be effectively co-deposited or serially deposited with the polymer. Drugs and other bioactive agents can be attached to the polymer either before or after deposition. When co-deposited with a polymer and depending on the polymer, the agent can be released or eluted from the polymer matrix in a time-dependent manner. Controlled time release profiles can be developed for agents deposited in combination with a coating polymer.

Accordingly, the invention provides a method to efficiently attach polymers to uniquely spinulose substrate surfaces. The nanostructured surfaces exhibit excellent adhesion and durability, while avoiding use of complicated, hazardous and inefficient chemistry; e.g., the silane, photo-, thermo-couplings conventionally used for polymer attachment, to an underlying substrate, as well as ultraviolet and heating steps that may cause surface damage. An additional advantage of the invention is the option to use polymers with functional groups, in effect providing an additional functional feature to the surface without employing additional steps to modify the deposited polymer.

The polymer films deposited on metal spinulose surfaces are highly resistant to shear and thermal peeling. Compared to polymer coatings on smooth or roughened surfaces such as metal surfaces, polymer coatings cannot be removed in comparable pull tests.

An advantage of preparing polymer surface films on spinulose metal surfaces is the application of many types of polymers to spinulose metal surfaces by any of a number of application methods. A simple dipping procedure can be used, which is rapid and inexpensive compared to other surface coating methods, including spraying, casting, spin coating and plasma deposition.

Several types of polymers can be polymerized on the spinulose metal surface, including thermosetting polymers, polymerized from monomers requiring either low or high polymerization temperatures. A spinulose surface, for example, can be contacted with either low or high polymerization temperatures as required for many thermosetting polymers. High polymerization temperatures can be employed without significant changes to a spinulose metal surface, such as Ti which has a melting temperature of over 1000° C. Photopolymerizable molecules requiring use of ultraviolet light or other radiation also do not affect the underlying spinulose metal surface. A wide range of polymers are suitable for coating on spinulose metal surfaces. Thus a significant advantage of the spinulose metal surfaces preservation of surface structure and binding properties even when heating is required to cure or polymerize a precursor monomer.

There are several advantages to polymer films that are strongly and durably adhered to surfaces with spinulose surface features. Biodegradable, biocompatible polymers can serve as a diffusion barrier against a reservoir device; e.g., silver oxide, to control release rate. A semi-permeable membrane over a drug-loaded surface with select polymer/copolymers can be fabricated to meet specific functional requirements. Similarly, a drug can be loaded onto a spinulose metal surface or polymer coated spinulose surface and used to create a controllable drug delivery system; for example using a biodegradable polymer(s)/co-polymer(s) for controlled release. Alternatively, a bioactive agent can be dispersed or dissolved in an inert polymer that is then cast or sprayed on a spinulose metal surface.

Functional polymers can also be used. Examples include monofunctional or bifunctional thiol, amino, maleimidyl, p-nitrophenyl, carboxyl, aldehyde active and/or N-hydroxysuccimidyl activated ester PEG polymers or any polymer derivative, and the like, adhered to a spinulous surface which can serve as a platform for attachment of biological molecules. Depending on the choice of polymer, one can introduce other desirable characteristics to the substrate surface. Examples include conjugation of biomolecules to the active sites of a dicarboxylic acid-PEG while simultaneously utilizing the PEG chain of the same molecule for protein passivation; improving cell adhesion by introducing not only an underlying nanostructured surface, but also a nanostructured surface topically modified with a biological polymer, such as collagen fibronectin, vitronectin, laminin and the like.

An additional feature of the metal spinulose surfaces is the ability to attract several types of cells, including fibroblast cells, including human skin fibroblast cells, human gingival fibroblast cells and human periodontal ligament fibroblast cells, as well as osteoblast cells and human umbilical endothelial cells. The disclosed spinulose surfaces are useful in implant devices where adhesion and proliferation of cells are important in bone or other types of restoration.

As discussed, the use of medical implant device, and particularly, miniaturized devices used to sense, pace and deliver therapy to the heart has become a necessity in the medical device field due to the intricate pathways of the pulmonary system. There is a need to reduce the size of electrodes that come into contact with the heart, not only for increased in vivo compatibility but also for creating a more effective implant. Unfortunately, by reducing the electrode size and ultimately the area on conventionally used heart implanted electrodes devices, the impedance increases to unacceptable levels for in vivo use. The present invention provides surfaces on such specialized electrodes that have higher conductance for the same area by increasing the capacitance of the interface, thereby decreasing electrode impedance.

### DEFINITIONS

“Spinulose” as defined in the Random House Unabridged Dictionary refers to a spiney appearance and in Webster's New International Dictionary Third Edition, as “covered with small spines”. Spinules, or small or minute spine, are distinguished in appearance from larger, more hair-like appendages commonly characterized as whiskers or columnar structures and which are typically wire or rod-like in appearance.

Spinulose metal surfaces, as described herein, are produced under special nano plasma deposition conditions. The surfaces are unique in appearance, showing distinctly pointed spikey projections over the surfaces. The projections are randomly spaced on round nanoparticulates, ranging in size from 1 μm up to about 50 μm in diameter. The projections appear as small spines on an irregularly rounded mass similar to a rough or prickly envelope of some fruits.

As used herein, “substantially” is intended to indicate a limited range of up to 10% of any value indicated.

As used within the context of the claimed subject matter, the term “a” is not intended to be limited to a single material or element but may indicate a collection or number of a material or element.

Physical vapor deposition (PVD) is used to describe a class of processes that involve the deposition of material, often in the form of a thin film, from a condensable vapor which has been produced from a solid precursor by physical means. There are many ways of producing the vapor, and many modifications to each of these processes. Examples of PVD processes include evaporation, sputtering, laser ablation and arc discharge. PVD can involve chemical reactions, such as from multiple sources, or by addition of a reactive gas.

Electron beam evaporation is use of an electron beam to heat a metal so that it evaporates. The vapor can be deposited on a surface.

Chemical vapor deposition (CVD) is the growth of material from a gas phase precursor, due to reaction or reactions that often occur on a surface. The reactions are frequently promoted by using an elevated substrate temperature. Alternatively the reactions can be achieved by enhancing the reactivity of the precursors using a plasma (PECVD) or hot wire.

Atomic layer deposition (ALD) is a CVD method involving growing materials by pulsing multiple precursors that react with a surface in a self-limiting manner.

Biomolecules are agents or materials that have some biological interactions; e.g., drugs, proteins, cells and bioorganisms such as bacteria and viruses.

### BRIEF DESCRIPTION OF THE DRAWINGS

FIG. 1 is a sketch of a typical ion plasma deposition apparatus showing a pure metal cathode target 1; substrate 2; substrate holder 3; vacuum chamber 4; power supply for target 5; and arc control 6. Not shown is an inlet into the vacuum chamber 4 for introducing a gas flow, which may be an inert gas, or reactive gas such as oxygen.

FIG. 2 is an FEG-SEM image of a spinulose titanium coating deposited from a titanium plasma at an angle of θ_{c}=0° with a θₛ of 0° on a 316L stainless steel or titanium substrate.

FIG. 3 is a FEG-SEM image of an oblique spinulose titanium coating deposited from a titanium plasma at an angle of θ_{c}=0° with a θₛ of 45° on 316L stainless steel.

FIG. 4 is a FEG-SEM image of a spinulose titanium coating deposited from a titanium plasma at an angle of θ_{c}=80° with a θₛ of 0° on 316L stainless steel.

FIG. 5 is a FEG-SEM image of a round particulate titanium coating deposited at an angle of angle of θ_{c}=0° with a θₛ of 0° on a 316L stainless steel or titanium substrate.

FIG. 6 is an FEG-SEM image of a spinulose zirconium coating formed from a zirconium plasma at an angle of θ_{c}=0° with a θₛ of 0° on a 316L stainless steel or titanium substrate.

FIG. 7 is a FEG-SEM image of an aluminum geometric coating deposited from an aluminum plasma deposited at an angle of θ_{c}=0° with a θₛ of 0° on 316L stainless steel.

FIG. 8 is a FEG-SEM image of Ag/AgO deposited by nanoplasma deposition onto a spinulose titanium surface on a titanium substrate

FIG. 9 is an FEG-SEM image of PLLA coated spinulose titanium scratched with a conospherical scratch probe with increasing normal load.

FIG. 10 is an FEG-SEM image of PLLA coated on smooth titanium scratched with a conospherical scratch probe with increasing normal load.

FIG. 11 is an elution profile of silver from Ag/AgO deposited on a spinulose titanium surface without a PLLA polymer coating (o) compared with silver eluted from Ag/AgO coated on a spinulose titanium surface with PLLA polymer coating (x). Elutions were performed in phosphate buffered saline (1×PBS) and mL/cm² [Ag] measured by ICP.

FIG. 12 shows human periodontal ligament fibroblast cell adhesion on titanium coated with titanium spinulous coating from a titanium plasma deposited at an angle of θ_{c}=0° with a θₛ of 0° compared to a titanium round coating from a titanium plasma deposited at an angle of θ_{c}=0° with a θₛ of 0° and no coating.

FIG. 13 is a FEG-SEM image of a titanium spinulose coating formed from a titanium plasma deposited at an angle of θ_{c}=0° and θₛ of 0° on 0.0005″ diameter type 303/304 stainless steel wire used to similate a miniaturized electrode.

FIG. 14 is a sketch of the electrode apparatus for taking impedance measurements of spinulose coated and bare electrode using an MCP-BR2822 portable LCR meter (A).

FIG. 15 is the impedance modulus of spinulose and unmodified 0.005″ stainless wire electrodes ˜250 um length, measured in 0.45% (w/v) NaCl_{(aq)} at room temperature.

### DETAILED DESCRIPTION OF THE INVENTION

Nanoplasma deposition (NPD) is a vapor deposition method that has been modified to produce uniquely nanotextured spinulose metal surfaces and films. The surfaces are stable, provide a strong matrix for cell attachment and growth, and are highly adherent to polymer coatings. Polymer surface coatings over the spinulose metal surfaces retain the spinulose surface nanofeatures and offer an additional platform for incorporating dual functionality onto substrate surfaces, either as attachments to the polymer itself or as overlying protective coatings.

The spinulose surface features of NPD deposited metals contribute to reaction with external environments and to binding with other materials. Adherent polymer coatings and films on spinulose surfaces make it possible to protect a metal substrate from external forces and/or to endow a substrate surface with functional or linking groups suitable for attaching biomolecules such as drugs. Polymers of many different types are suitable for applying to a NPD spinulose nanoparticulate surface, including hydrophilic, hydrophobic and functionalized polymers. PLLA coated spinulose titanium, for example, exhibits strong adhesion compared to the poor adhesion of PLLA coated over smooth titanium.

Polymer coatings may act as time release barriers for selected bioactive agents, particularly those used on medical devices. In an illustrative example, poly-L-lactic acid (PLLA) was tested because of its biocompatibility and potential application for coatings on stents, guidewires and various implants. PLLA and poly(lactic-co-glycolic acid) (PLGA) coatings were applied as diffusion barriers over reservoirs of Ag/AgO deposited on spinulose titanium substrates. Silver released from surface-deposited Ag/AgO is recognized as having antimicrobial properties and has been used as an antimicrobial agent externally and as a coating on implanted or internally used devices.

Nanotextured spinulose metal surfaces can be produced by controlled NPD of a metal on a wide range of substrate surfaces. Nanoplasma deposited titanium and/or zirconium, for example, deposited under the specific conditions described herein exhibit features significantly different in appearance and properties from conventionally vapor deposited metals and metal compounds. A nano-rough surface initially appears as spikes on round particulates, growing into a spiky or spinulose surface when the deposition is cycled under controlled conditions.

The deposition method of the invention is a modified ion plasma deposition process in which a plasma is generated from a metal target and deposited onto a substrate in a controlled atmosphere environment under reduced pressure. The nano plasma deposition process (NPD) is basically a vacuum deposition of ionized material generated as a plasma by applying voltage and current to a cathode target such that ionized particles are deposited on a substrate. The metal plasma initially deposits as nanoparticulates, atoms and ions, which after further deposition under the described controlled deposition cycling conditions will form a unique nanostructured surface. Unique surface features of the deposited metals are formed under vacuum and/or in an inert atmosphere, typically an inert gas such as argon, using a cycling process. The presence of oxygen or nitrogen may result in formation of metal oxides or nitrides, resulting in surface features different from the nanostructures formed from deposition of substantially pure metals.

The spinulose nanostructured surfaces produced under defined NPD deposition conditions have been produced with commercially pure titanium (grade 2) and with zirconium, the latter containing up to 4.5% hafnium in some samples. Using the particular described cycling deposition method, spinulose-type surfaces were not observed with aluminum, cobalt, copper, nickel, (pure) hafnium, 316L stainless steel, nitinol, silver or titanium 6-4 deposited from metal targets on stainless steel substrates. On the other hand, in some cases, these metals form other types of unusual nanostructured surfaces, which are different from the spinulose appearance of deposited titanium or zirconium. Generally, with the exception of aluminum, the nickel, cobalt, copper, silver, hafnium, 316L stainless steel, nitinol and titanium 6-4, the nanostructured surfaces are basically globular or stacked globular in shape.

NPD aluminum surfaces are markedly different from Ti and Zr and the other metals cyclically deposited NPD metals. Pure aluminum metal deposited under the same conditions described for titanium and/or zirconium has a stacked appearance with a geometric cube-like structure different from the structures observed with titanium and other metals. While spinulose surfaces for aluminum and other metals are not observed under the conditions used to produce spinulose titanium or zirconium nanostructures, it may be possible to generate surface spinules by using modifications of the disclosed deposition procedures, such as, but not necessarily limited to, longer intervals between deposition cycles, distance from target and chamber pressure.

Spinulose nanostructured titanium and zirconium surfaces can be formed as coatings or films on virtually any metal, plastic, ceramic or glass substrate surface, including stainless steel, titanium, CoCrMo, nitinol, glass or silicon, as well as on silicone, poly(methylmethacrylate) (PMMA), polyurethane (PU), polyvinyl chloride (PVC), polyethylene terephthalate glycol (PETG), polyetheretherketone (PEEK), polytetrafluoroethylene (PTFE), polyethylene terephthalate (PET), ultra high molecular weight polyethylene (UHMWPE), and polypropylene (PP). Other metals, including aluminum, gold, platinum, copper and silver are also suitable substrates.

The process for producing spinulose Ti and Zr surfaces and the globular type surfaces observed with other metals, except aluminum utilizes a nano plasma deposition method comprising generation of a plasma from a metal cathode. Distance of the deposition target from the substrate can affect the nanostructural features of the deposited metal and surface coverage and can be adjusted to the particular apparatus configurations and deposition conditions. The substrate is housed in a vacuum chamber and, while the base pressure does not appear critical for spinulose Ti formation, the selected pressure, gas flow, cycling time, distance from the cathode and other parameters can influence properties such as spinulose height and surface density.

Deposition is preferably conducted in an inert gas atmosphere, e.g., argon, in order to avoid any chemical reaction with the metal being deposited. Titanium will react with some gases; for example, when nitrogen is present in the system, TiN may form. The deposited TiN is not spinulous; rather, as reported by others using conventional vapor deposition, the nanostructured Ti surface typically has projections that are more whisker-like or column-like in appearance. In an exemplary NPD method for producing Ti spinulose surfaces, an argon atmosphere is employed, care being taken to use gas of high purity so that trace components do not react with the ionized titanium or other metal produced in the plasma.

Deposition is performed using a periodic deposition or cycling process. Gas flow and plasma discharge into the vacuum chamber are allowed to progress for a specified period of time. Gas flow is then reduced to near zero or, alternatively stopped completely, and plasma discharge is discontinued for a defined period of time before the cycle is reinitiated. The cycling is an unusual step and is believed to be important in producing the observed Ti and Zr spinulose structures. Images of initially deposited Ti or Zr show that the metal ion plasma first deposits as substantially round nanoparticles. With additional cycling, the particulates develop a more spike-like structure with increasing surface coverage as the number of cycles is increased.

In the examples shown, and using a small-scale apparatus, about 3-9 cycles are typically run with about 5-15 min of deposition followed by about 5-90 min of “resting” when gas flow is reduced or stopped and plasma generation is stopped completely. In the examples presented, distinct Ti spinulites were not observed until after about 3 cycles. More than 9 cycles generally increased the number and density of Ti and Zr spinulites, which can increase available surface area for attachment of biomolecules and/or drugs as well as increase coverage of substrate surface up to 85% or greater.

The morphology of the NPD spinulose coatings depends on the angle between the cathode surface normal and the direction of the substrate (θ_{c}) and also the angle between the depositing flux and the substrate surface normal (θₛ). Generally the most pronounced spinulose morphology can be produced with θ_{c}=0° and with a θₛ of 0°. As θₛ is increased from 0° the structure of the spinulose coating grows more anisotropic. At oblique incidence angles the spinulites tend to grow such that they point away from the depositing flux. This is opposite to the direction observed in GLAD (glancing angle deposition) others have reported (U.S. Pat. No. 6,248,422). Spinulose Ti surfaces are obtained from depositions with a flux θ_{c}=0° and θₛ ranging from 0° to 80° and with a flux θₛ=0° and θ_{c} ranging from 0° to 80°.

In order to prepare surfaces for attaching polymer coatings, conventional texturing techniques such as sandblasting have often been used by others to improve polymer adherence. Yet lack of polymer adherence remains a concern. The nanostructured spinulose surfaces of the present invention are distinctly different from whiskered type metal surfaces, columnar types of thin film surfaces and from the intergranular etched polymer surfaces to which an immersion plated metal is applied for the purpose of increasing peel strength. The spinulose surfaces, when coated with a polymer, can be used for controlled release of bioactive or other agents.

Titanium spinulose surfaces on a metal, polymer, ceramic or glass substrate surface are highly nanostructured, but maintain basic structure when coated with Ag/AgO, polymers, or thin layers of drugs/biomolecules, as can be seen from the SEM images.

To produce an adherent polymer coated surface, a substrate surface is first modified with nano plasma deposited (NPD) titanium or zirconium nanoparticulates, followed by application of the polymer onto the spinulose nanoparticulate surface. Depending on the polymer, the application may be by casting, spraying, dipping, electrospinning, or similar methods. In some applications, it may be advantageous to apply a polymer by vapor deposition, such as a plasma-enhanced chemical vapor deposition. Some monomers may polymerize on the spinulose surface and can be used to form very thin films.

Tape tests have confirmed that the adherence of polystyrene (PS), poly(lactic-co-glycolic acid) (PLGA), poly-L-lactic acid (PLLA) and polyethylene glycol (PEG) polymers to spinulose nanostructured titanium surfaces is surprisingly high and significantly better than adherence to smooth titanium surfaces. PS, PLGA, PLLA and PEG coatings were applied to spinulose titanium substrates as well as to smooth titanium surfaces in order to compare adhesion. Adhesion was determined by using the tape test as specified in ASTM D3359-08. This standard practice demonstrated that several polymer coatings with a range of chemical properties tightly adhered to spinulose nanostructured surfaces but failed to remain completely intact on a smooth titanium surface.

Several PLLA films were coated onto Ag/AgO previously deposited by vapor phase onto a smooth titanium surface. In all tests, the polymer coating sloughed from the metal surface. Ag/AgO was then vapor phase deposited onto a highly spinulose nanostructured titanium surface. Tests showed that the PLLA adhered well to the surface, in contrast to the lack of adherence on smooth titanium (unstructured surfaces).

In separate tests, Ag/AgO coated spinulose surfaces were coated with several polymers to determine the ability of the polymer to act as a protective coating and, importantly, whether or not a polymer film could act as a controlled release coating.

Polymers coated on the spinulose surface or polymer coated on Ag/AgO deposited on a spinulose surface remained strongly attached and showed little sloughing in solution or under mechanical stress.

Controlled release of polymer-coated silver has been demonstrated. Polymer coatings over Ag/AgO coatings on a titanium spinulose surface showed that PLLA and PLGA will sustain the release of silver over at least several days, while simultaneously maintaining polymer integrity on the surface. The examples illustrate that selected polymer coatings over bioactive agents and/or biomolecules deposited on spinulose titanium surfaces do not peel or slough from the surface and, importantly, can be used for timed or controlled release. In addition to Ag/AgO release, it is expected that other drugs, and bioactive molecules, including metals and metal containing compounds, can be attached or deposited onto a spinulose nanostructured surface, coated with a suitable polymer and used to obtain a desired time release profile.

Nanostructured spinulose metal surfaces act as scaffolds for polymer surfacing or for molecules initially deposited onto such spinulose surfaces. In certain embodiments, biomolecules and/or bioactive agents such as drugs and antimicrobials, e.g., silver, are deposited on the spinulose surface by nano or molecular plasma deposition, or by other conventional and well-known deposition methods, such that the nanostructure of the spinulose surface is preserved. In the example of Ag/AgO plasma vapor deposition (IPD) on a spinulose titanium surface, the SEM photograph indicates that the titanium spikes appear coated but otherwise retain a similar nanorough spike-like structure, by comparison with the SEM photograph of uncoated spinulose titanium.

Adhesive properties of PLLA on a spinulose nanostructured titanium surface are enhanced as demonstrated when using a conspherical scratch probe with increasing load normal to test the interfacial adhesion of PLLA to the spinulose nanostructured titanium substrate. The PLLA coating displayed good adhesion even around the severely damaged areas. In contrast, the same test with PLLA coated smooth titanium results in delamination of a region around the load, causing buckling and cracking of the polymer film.

The impedence of spinulose titanium coated stainless steel wires useful as leads or electrodes was compared with impedence of bare (uncoated) stainless steel wires. The spinulose titanium coated electrodes with a greater surface area than the bare wires, had a lower impedance over all frequencies measured. Such spinulose titanium coated wires and leads can be used in the fabrication of miniaturized devices used in a variety of medical implants.

### EXAMPLES

### Materials and Methods

Human Osteoblast cells (CRL-11372) were purchased from American Type Culture Collection (Rockville, Md.) as frozen cultures in complete media: 1:1 Ham's F12 medium and Dulbecco's modified Eagle's medium without phenol red with 2.5 mM L-glutamine, 10% FBS and 0.3 μg/ml G418. Before use, the vials were thawed, centrifuged and the cells resuspended in complete media before transfer into a culture device and incubated at 34° C. in 5% carbon dioxide. The cells were then subcultured in complete media after treating with trypsin-EDTA at either 34° C. or 39° C. Doubling time was 36 hr at 33.5° C. and 96 hr at 38.0° C. If not used immediately, the cells were stored frozen in complete media with DMSO added to each vial.

Human fibroblast cells (CRL-1502) were purchased from American Type Culture Collection as frozen cultures in complete media containing Eagle's minimal essential medium with Earle's BSS and 2 mM L-glutamine (EMEM) modified to contain 1.0 mM sodium pyruvate, 0.1 M non-essential amino acids, 1.5 g/L sodium bicarbonate supplemented with 10% FBS and 10 U/mL penicillin/streptomycin.

Human endothelial cells were purchased from VEC Technologies (Rensselaer, N.Y.) as frozen cultures in MCDB-131 media.

Cell sample vials were thawed at 37° C., centrifuged and the cell pellet resuspended in complete media before transfer to a culture device and incubated at 34° C. in 5% carbon dioxide. Cells were subcultured by rinsing and adding trypsin-EDTA before culturing in complete media and incubating at 34° C. or 39° C. If not used immediately, the cells were rinsed and stored in liquid nitrogen after addition of 10% FBS and DMSO to the vials.

Cathode material was titanium or zirconium 702 (UNS R60702 containing up to 4.5% hafnium).

### Example 1

### Spinulose Titanium or Zirconium Surfaces

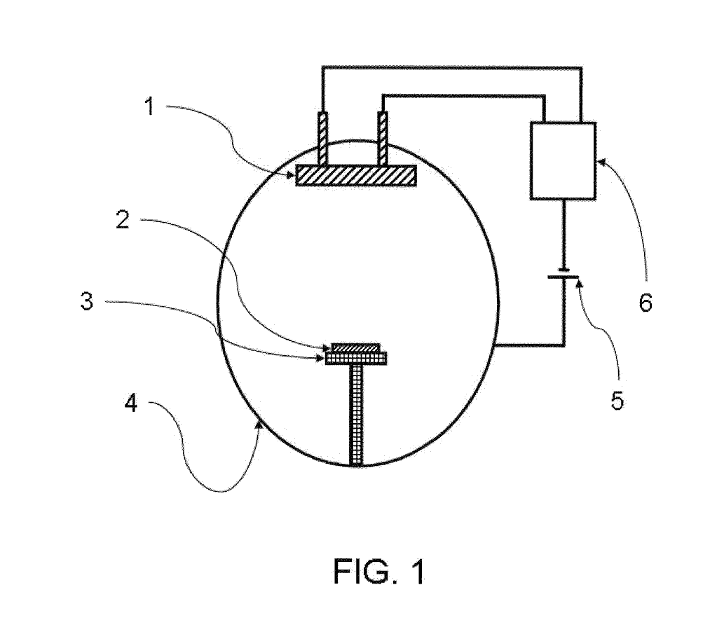

Nanostructured spinulose titanium or zirconium surfaces can be produced by a modified cyclic plasma arc deposition procedure termed nanoplasma deposition (NPD). The vapor deposition apparatus for producing the metal ion plasmas can be used both for conventional metal plasma deposition and the cyclic modification used to produce spinulose surfaces and films. The apparatus is shown in FIG. 1. The metal cathode targets are disposed in a vacuum chamber. An inert gas, typically argon, is not required but may be introduced into the evacuated chamber and deposition commenced. The substrate 2 is generally positioned 6-28 inches from the target and deposition is conducted intermittently for periods of approximately 1-20 minutes. During the intervals between depositions, there is no plasma discharge and the inert gas flow optionally can be reduced or stopped completely if desired. The intervals between depositions can be varied and are about 5-90 min with a typical run of about 3-27 cycles.

In several variations of deposition conditions for titanium on silicon or titanium substrates, argon was typically used as an inert gas at 100 sccm, depositions of 5 min at 90 min rest intervals, 9 cycles, 300 amps and 13 inches from the cathode. Surface coverage with spinulose titanium typically ranged from 85-98%. Distance from the cathode was also varied from 8-13 in a number of runs with θ_{c} ranging from 0° to 80°, gas flow 100-300 sccm and variation of rest interval from 5-90 min. Over two hundred variations were run, showing that these parameters can to varied to control surface coverage and spinule height, which ranged from over 1 μm to less than 0.2 μM.

Spinulites could be produced by this method on any of a number of substrates, including stainless steel, nitinol, CoCrMo alloy, silicon, titanium, anodized titanium, glass, silicone, poly(methyl methacrylate) PMMA, polyurethane (PU), polytetrafluoroethylene (PTFE), polyvinyl chloride) (PVC), polyethylene terephthalate (PET), ultra high molecular weight polyethylene (UHMWPE), polyethylene terephthalate glucol (PETG), polyetheretherketone (PEEK) and polypropylene (PP).

The selected substrate is ultrasonically cleaned in detergent (ChemCrest #275 at 160° F.), rinsed in deionized water and dried in hot air prior to the deposition process. The clean substrate is then placed in the chamber and exposed to nano-plasma deposition (NPD) using the special deposition conditions described. The cathode is commercially pure titanium cathode (grade 2) or zirconium 7021. The substrate is mounted in the vacuum chamber at distances from 6-28 in from the cathode (measured from the centre of the cathode). The angle between the substrate surface normal and a line from the centre of the cathode to the substrate, θc, can be varied in the range of 0-80°. The angle between the depositing flux and the substrate surface, θs, is varied in the range of 0-80°. The chamber is pumped to a base pressure of between 1.33 mPa-0.080 mPa. The arc current is varied from a 15-400 A with an argon burn pressure of 0.1-5.5 mT.

The process is run in cycles, with each cycle consisting of plasma discharge intervals (varied over the range 1 to 20 minutes) followed by intervals where there is no discharge and or gas flow (between 5 and 810 minutes), except that gas flow can be optionally maintained. Each process consisted of 3-27 cycles.

Following plasma deposition, the samples are characterized by scanning electron microscopy (SEM). SEM images were obtained with a Tescan Mira Field Emission instrument (Brno, Czech Republic, Jihomoraysky, Kray) equipped with a SE detector, at a magnification of 5 K and 10 K times at 10 kV.

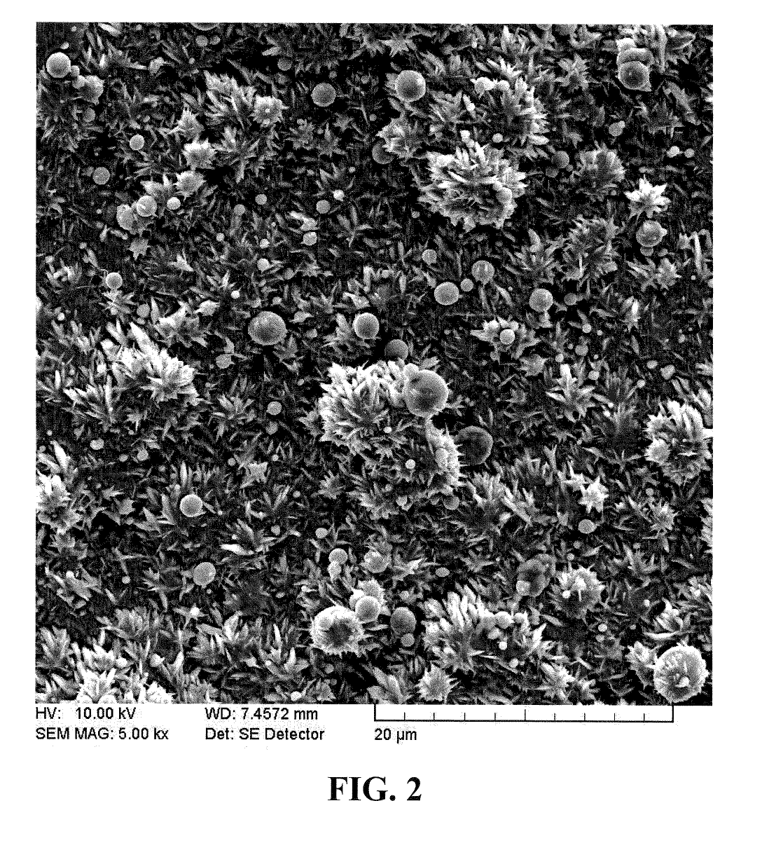

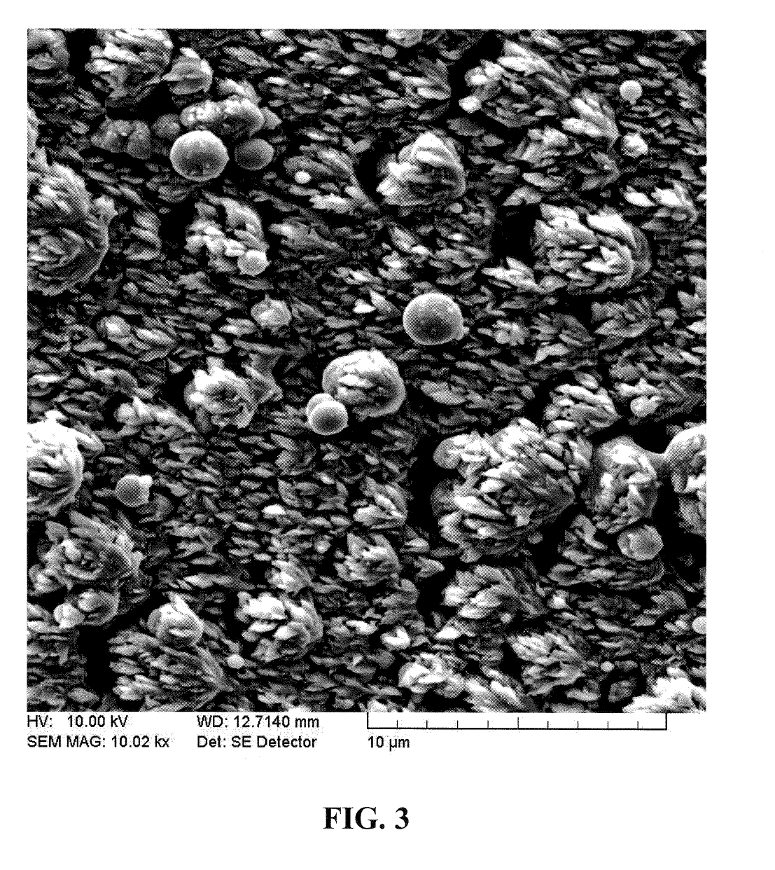

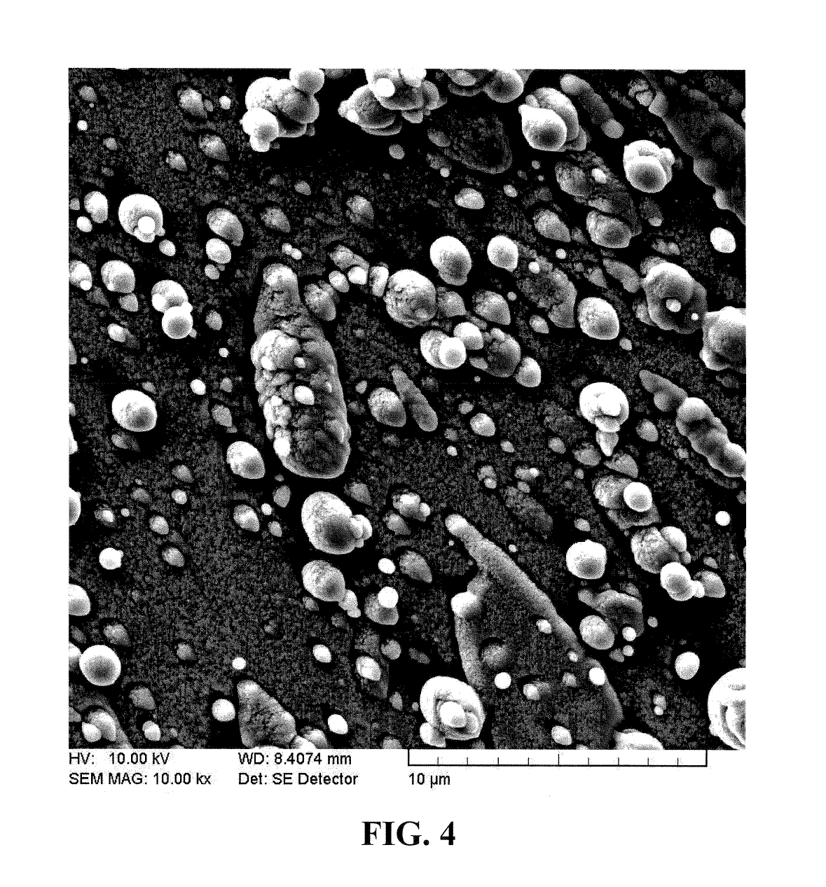

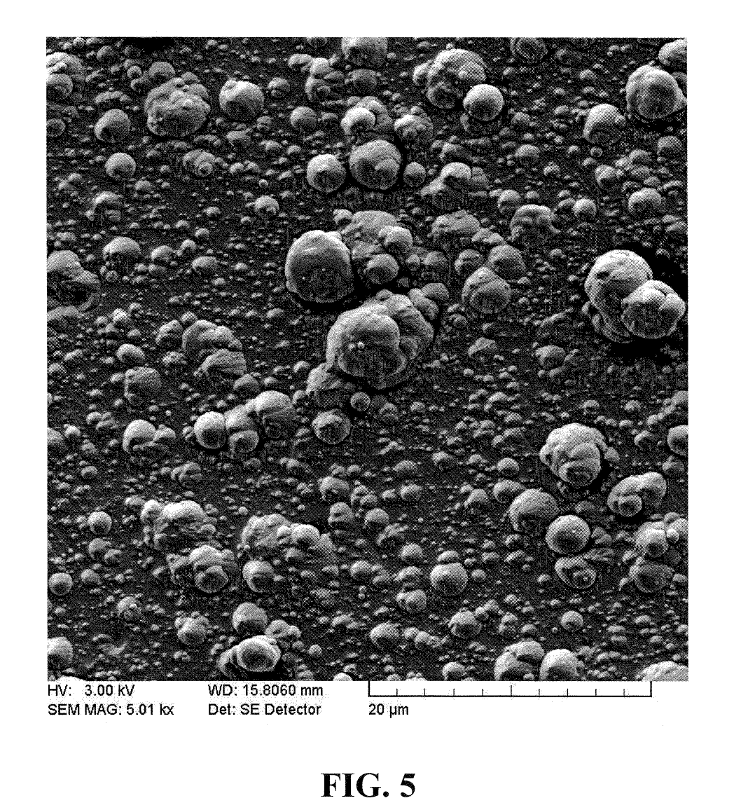

Initially NPD deposited particles from titanium or zirconium plasmas are typically round and will differ in size and distribution depending on power and/or time of deposition. Under the described specified deposition conditions, the titanium or zirconium metal particles develop nanosized spike-like protrusions, which were observed as spinules or small thorny spines as shown in FIG. 2 for titanium. FIG. 3 shows oblique titanium spinules resulting from a deposition angle θ_{C}=0° with a θ_{S} of 45°. Deposition with θ_{C}=80° with a θ_{S} of 0° exhibits much less spinulose character, FIG. 4. Conventional physical vapor deposition results in a round particulate titanium coating on a titanium or stainless steel substrate, as shown in FIG. 5.

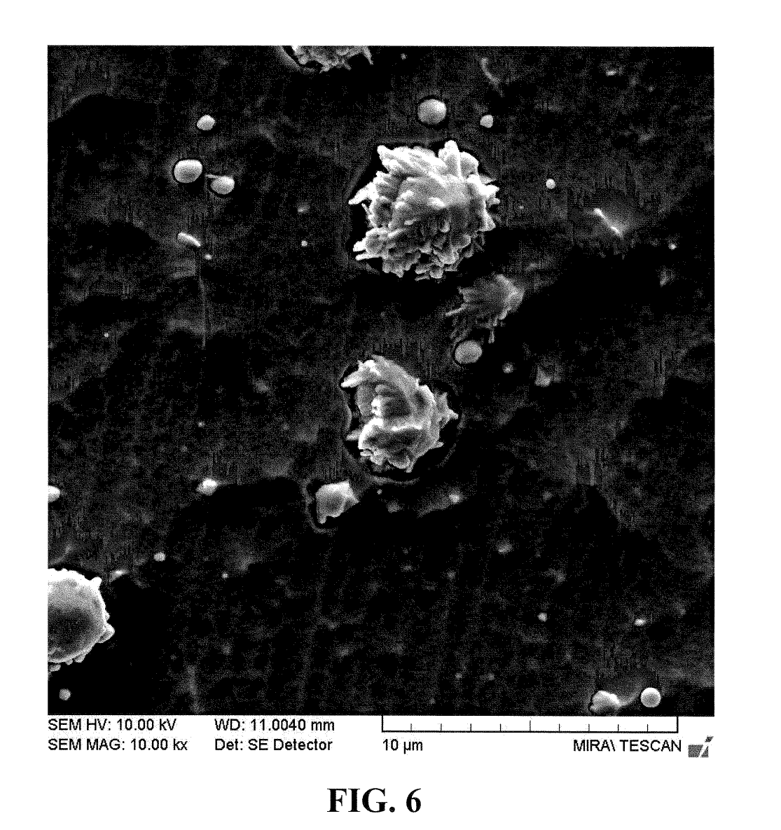

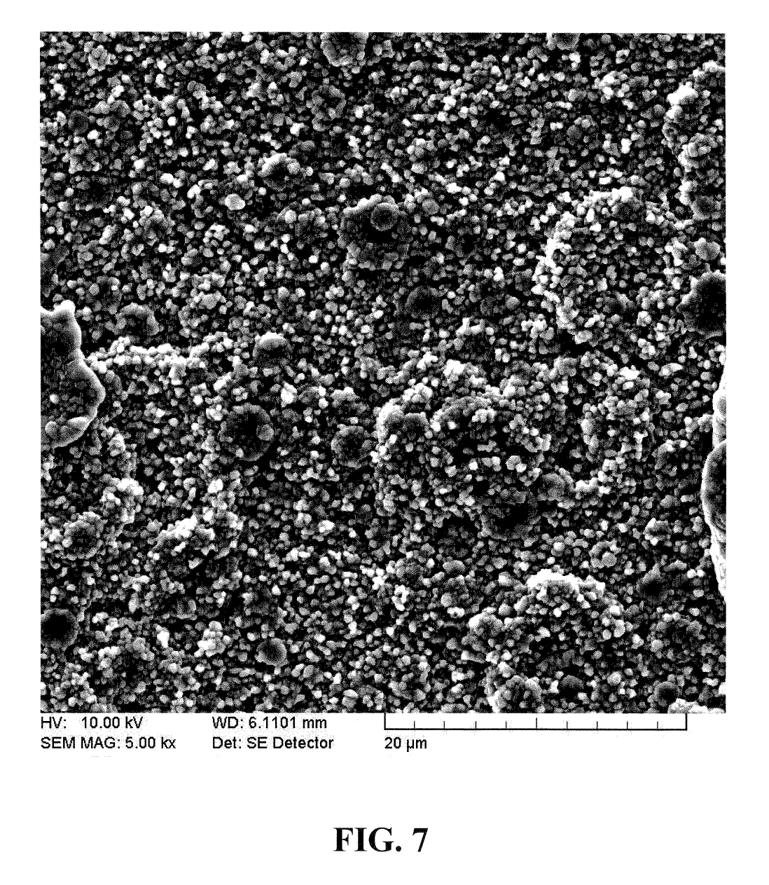

Spinulose surfaces under cyclic deposition conditions (NPD) similar to those used for Ti are observed for zirconium, FIG. 6. Aluminum does not produce a spinulose surface under these conditions although the surface image shows unusual nanostructural features, see FIG. 7, but lacks the spinules seen with titanium and zirconium.

### Example 2

### Polymer Coated Ionic Plasma Deposited (IPD) Silver/Silver Oxide

Ionic Plasma Deposition (IPD) creates a highly energized plasma from a target material, typically solid metal, from a cathodic arc discharge. An arc is struck on the metal and the high power density on the arc vaporizes and ionizes the metal, resulting in a plasma, which sustains the arc because the metal vapor itself is ionized, rather than an ambient gas.

The same apparatus used for the NPD process, FIG. 1, was used to control deposition of a silver/silver oxide plasma ejected from a silver cathodic arc target source 1 onto a substrate 2 within the vacuum chamber 4 or by a power supply 5 to the target and adjustment of arc speed 6. The closer a substrate is to the arc source, the larger and more densely packed will be the particles deposited on the substrate.

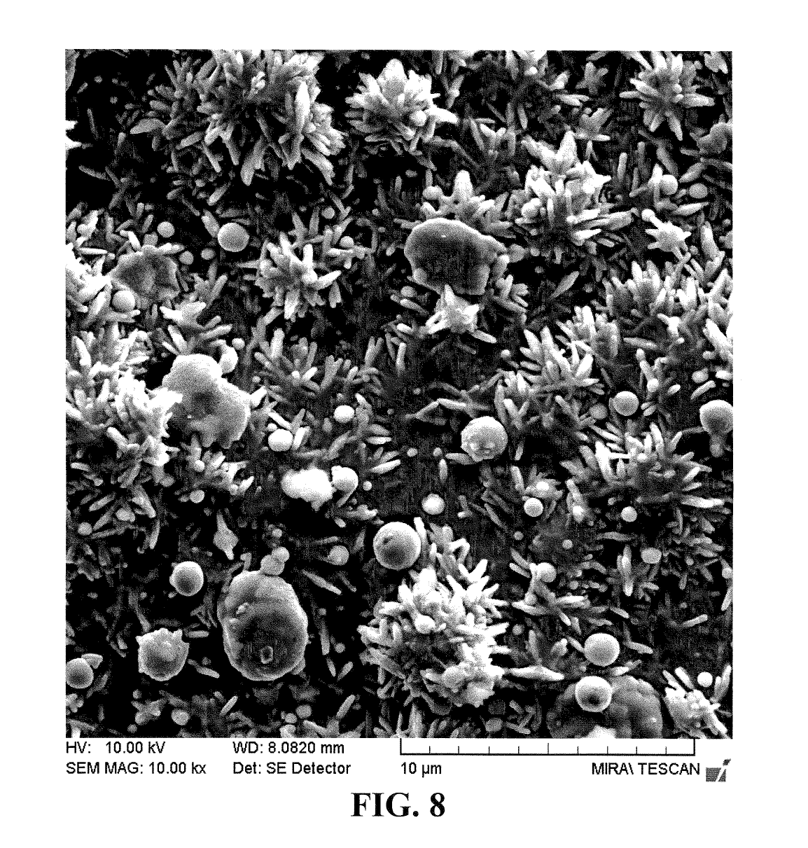

Ag/AgO was deposited onto a spinulose titanium surface coated on a titanium substrate. As shown by SEM in FIG. 8, the spinulose features of the titanium are maintained after Ag/AgO deposition.

A 4% w/v poly-L-lactic acid polymer (PLLA) solution in chloroform was cast from a pipette over the surface of a Ag/AgO deposited by IPD on a smooth titanium substrate. The polymerized coating was only weakly adherent to the underlying silver surface as evidenced by peeling of the film shortly after immersion in phosphate buffered saline (PBS) or deionized water at 37° C. in less than one day.

In contrast to Ag/AgO deposited on a smooth titanium surface, Ag/AgO deposited on spinulose titanium surfaces using IPD and coated with PLLA retained the PLLA coating.

### Example 3

### Polymer Adhesion to Spinulose Titanium Surfaces

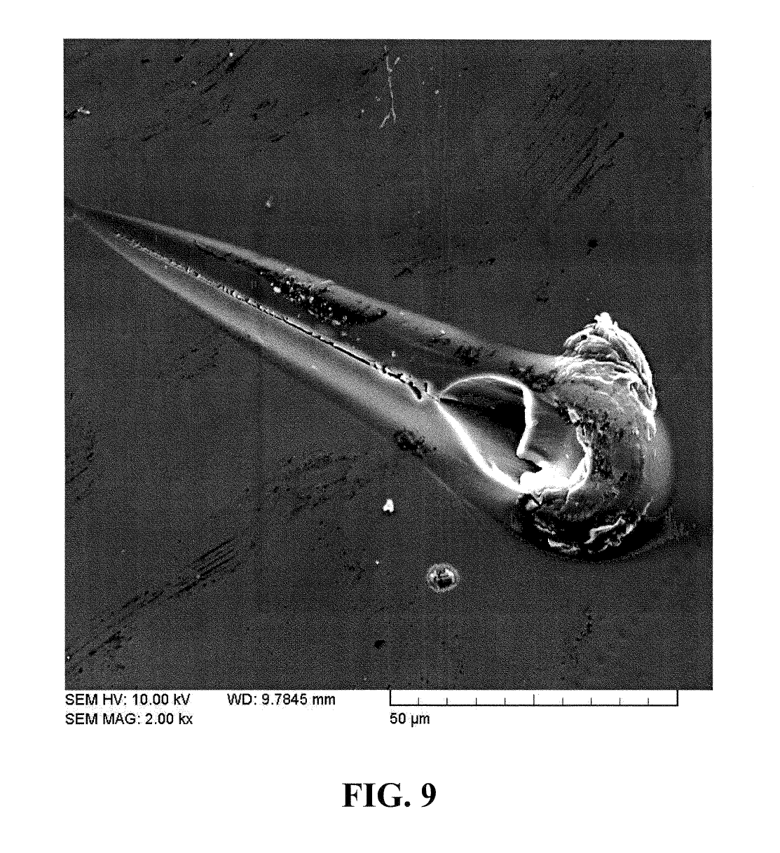

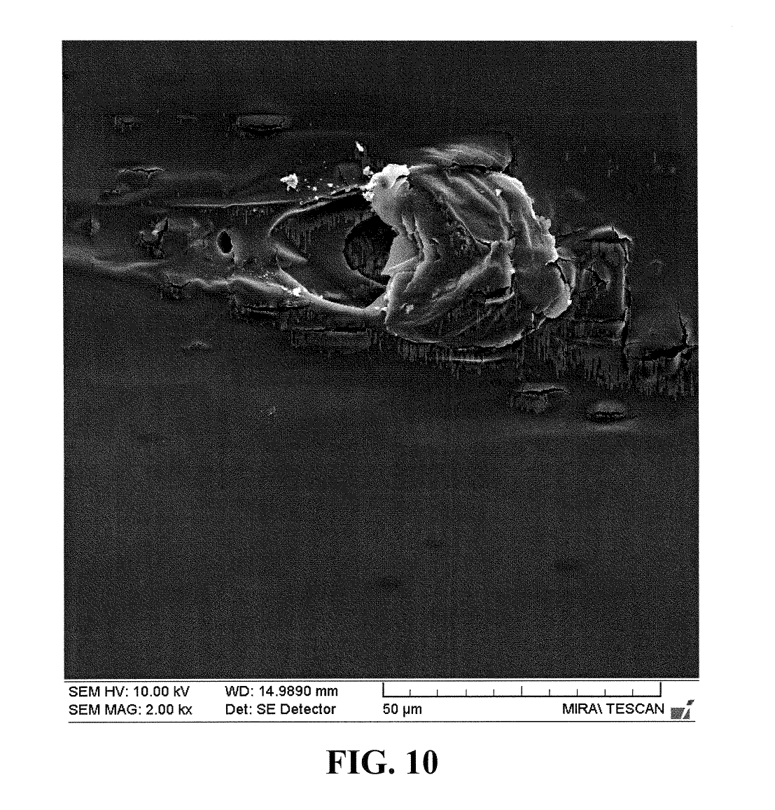

Interfacial adhesion of PS, PLGA, PLLA and PEG coatings to spinulose titanium and to smooth titanium surfaces were compared using a scratch induced delamination process. This test demonstrated that the polymer coatings with a range of chemical properties exhibited little, if any, delamination from the spinulose nanostructured titanium surface. The polymers were typically observed to fracture and in many cases fall off the smooth titanium surface. FIG. 9 shows the enhanced interfacial adhesion properties of PLLA to a spinulose nanostructured titanium surface following a scratch test compared to the poor adhesion properties of PLLA to the smooth titanium, FIG. 10. The work force in both scratch tests was similar.

The lack of delamination evident from observations with light microscopy showed that the interface is considerably toughened with the spinulose surface. The scanning electron microscopy (SEM) revealed a difference in failure modes, shown in FIG. 10, with the non-spinulose sample showing cracks in the polymer coating above regions subject to delamination that were not observed in the spinulose coated sample, FIG. 9.

### Example 4

### Elution of Silver from PLLA Coated Ag/AgO on Spinulose Titanium

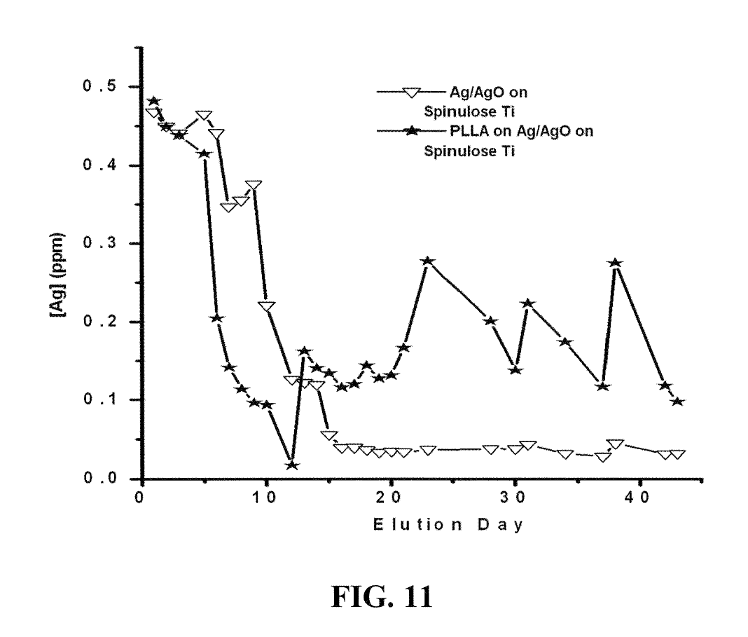

A spinulose titanium surface was formed on a smooth titanium substrate as described in Example 1. Ag/AgO was deposited on the spinulose surface by ion plasma deposition (IPD) from a silver cathode as described in Example 2 with use of a silver target. A film of PLLA was then cast over the Ag/AgO as described in Example 2. The coated Ag/AgO was placed in deionized water, physiological saline or PBS at 37° C. FIG. 11 shows an elution profile in PBS for silver after 43 days comparing silver profiles of PLLA coated Ag/AgO and uncoated Ag/AgO deposited on a spinulose titanium surface. At day 13 in the PBS, the Ag/AgO remaining on the PLLA coated spinulose titanium surface was higher than the amount deposited on the Ag/AgO spinulose titanium only surface. Even after soaking for at least 43 days in deionized water, the polymer film remained well adhered to the spinulose surface.

### Example 5

### Adhesion to Spinulose Titanium Surfaces

The adhesion or pull-off strength of RTV silicone adhesive to spinulose titanium, round titanium, roughened and smooth PEEK, 316L and titanium surfaces were compared using the PosiTest Pull-Off Adhesion Tester (DeFelsko, N.Y.).

A 0.5 cm drop of NuSil MED-1511 RTV Silicone Adhesive (NuSil Technology, CA) was applied to the surface of a 10 mm aluminum dolly. The dolly was adhered to the spinulose titanium surface and the adhesive allowed to cure. The pull-off strength as well as the weakest plane within the dolly/adhesive/coating/substrate system were measured using the adhesion tester. Table 1 shows the average PSI at which there was a fracture as well as where the respective fracture occurred in the system.

This test further demonstrated that the silicone adhesive exhibited little, if any, delamination or failure from the spinulose nanostructured titanium surface, while the adhesive typically observed to partially and/or completely delaminate from or partially and/or completely fail at the round titanium, roughened and smooth PEEK, 316L and titanium surfaces.

TABLE 1

| | | | Average |
|---|---|---|---|
| Substrate | Coating | Plane of Failure | PSI |
| PEEK | Untreated | Failure at substrate | 249 |
| PEEK | Roughened | Failure at substrate | 238 |
| PEEK | Round | Partial failure at substrate/ | 329 |
| | | dolly of up to 10%/90% | |
| | | Delamination at substrate of up | 346 |
| | | to 20% | |
| PEEK | Spinulose | Failure at Dolly | 388 |
| 316L | Untreated | Failure at substrate | 241 |
| 316L | Roughened | Failure at substrate | 320 |
| 316L | Round | Failure at substrate/coating | 262 |
| 316L | Spinulose | Failure at dolly | 399 |
| Titanium | Untreated | Failure at substrate | 328 |
| Titanium | Roughened | Partial failure at substrate/ | 373 |
| | | dolly up to 30%/70% | |
| | | Delamination at substrate up to 20% | 364 |
| Titanium | Round | Failure at substrate | 314 |
| Titanium | Spinulose | Failure at dolly | 364 |

### Example 6

### Periodontal Ligament Fibroblast and Gingival Fibroblast Cell Adhesion

Human periodontal ligament fibroblast and human gingival fibroblast seven day adhesion tests were carried out on titanium coated with a titanium spinulous coating formed from a titanium plasma deposited at an angle of θ_{c}=0° with a θₛ of 0° and compared with cell adhesion on titanium coated with titanium round coating formed from a titanium plasma deposited at an angle of θ_{c}=0° with a θₛ of 0° and with uncoated titanium.

The substrates were placed in wells using sterilized tweezers and exposed to UV light for one hour. The substrates were then rinsed with 2.0 mL of room temperature (1×PBS). The desired amount of room temperature Complete Media (supplemented with FBS and antibiotic) was added to each well. The cells were seeded onto the substrates at 3500 cells/cm² and incubated at 34° C., 5% CO₂ for seven days. Following incubation, the media and non-adherent cells were removed. The substrates were then rinsed with room temperature 1×PBS and fixed with 4% paraformaldehyde. The nuclei of adherent cells were fluorescently stained with Hoescht stain and counted using a fluorescent microscope.

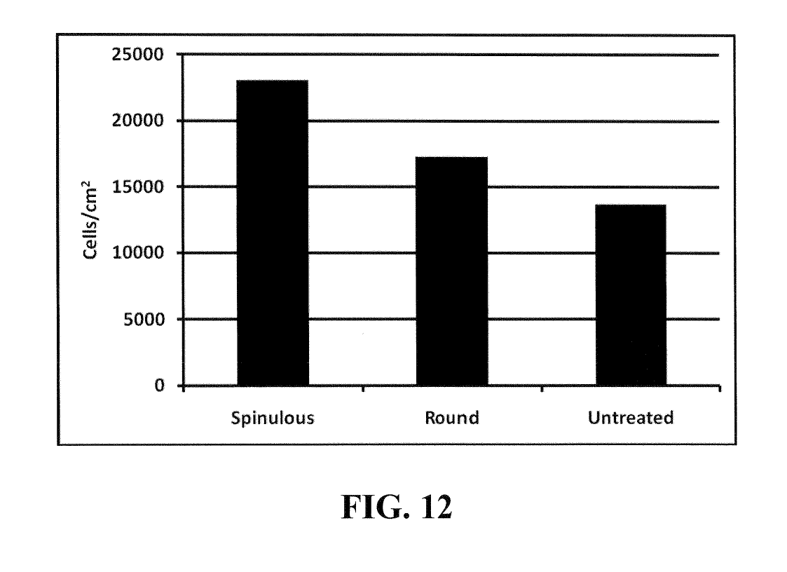

FIG. 12 compares the results of the human periodontal ligament fibroblast cell seven day adhesion tests on spinulous Ti, round Ti and untreated substrates. There is an increase in the number of human periodontal ligament fibroblast cells adhered to the surface of titanium coated with a titanium spinulous coating from a titanium plasma deposited at an angle of θ_{c}=0° with a θₛ of 0° over that of a titanium round coating from a titanium plasma deposited at an angle of θ_{c}=0° with a θₛ of 0° and no coating.

Similar results were obtained for human gingival fibroblast cell adhesion with approximately 1200 cells/cm² for spinulous Ti surfaces, 980 cells/cm² for round Ti and 800 cells/cm² for uncoated surfaces. Human osteoblasts showed increased cell adherence to spinulose Ti surfaces compared to uncoated surfaces, regardless of underlying substrate, including PVC, silicone, PU, PMMA, PET, UHMWPE and PTFE substrates.

### Example 7

### Reduction of Electrode Impedance Using Spinulose Titanium Surfaces

By reducing electrode area, the impedance of conventional electrodes increases to an undesirable level. This example shows that by applying a spinulose titanium surface to miniaturized electrode devices, the constraint of reducing electrode area characterized by increasing impedance can be eliminated. The spinulose titanium surface consisting of micron-sized high aspect ratio titanium structures, gives a higher conductive material area for the same geometric area. Thus by increasing the capacitance of the interface, the electrode impedance can be decreased.

The exposed end of 0.005″ diameter type 302/304 stainless steel (McMaster Carr 9882K11) wire was used to simulate a miniaturized electrode. Cut lengths of wire were sonicated in isopropyl alcohol for approximately 30 minutes and left to air dry. The cleaned wire was placed in the vacuum chamber on a floating holder with the exposed end of the wire placed approximately 13″ from the cathode at an angle of θ_{C}=0° with a θ_{S} of 0°. Prior to deposition, the chamber was pumped to a base pressure of at least 807 mPa.

The deposition was carried out in 5 minute intervals with 90 minute intervals of no arc current and no gas flow. This deposition-pause cycle was repeated 9 times. A 200 A arc discharge was generated in a background of 160 mPa of argon on a pure titanium (grade 2) cathode (20 in ×6 in).

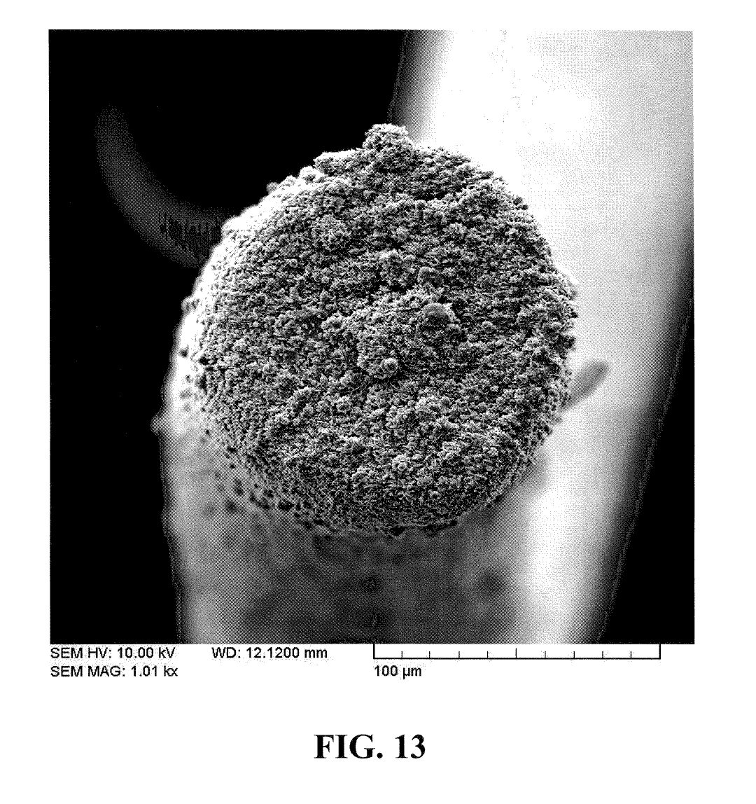

Samples were imaged in a FEG-SEM (Tescan Mira), operated with an accelerating voltage of 10 kV. The images showed a pronounced spinulose morphology (see FIG. 13) with the fraction of surface area covered by spinulose features estimated to be at least 85%.

As an insulating layer, clear nail polish (Sally Hansen) was used to coat the spinulose coated wire as well as uncoated wire, leaving the tips exposed. These coatings were applied using the included brush.

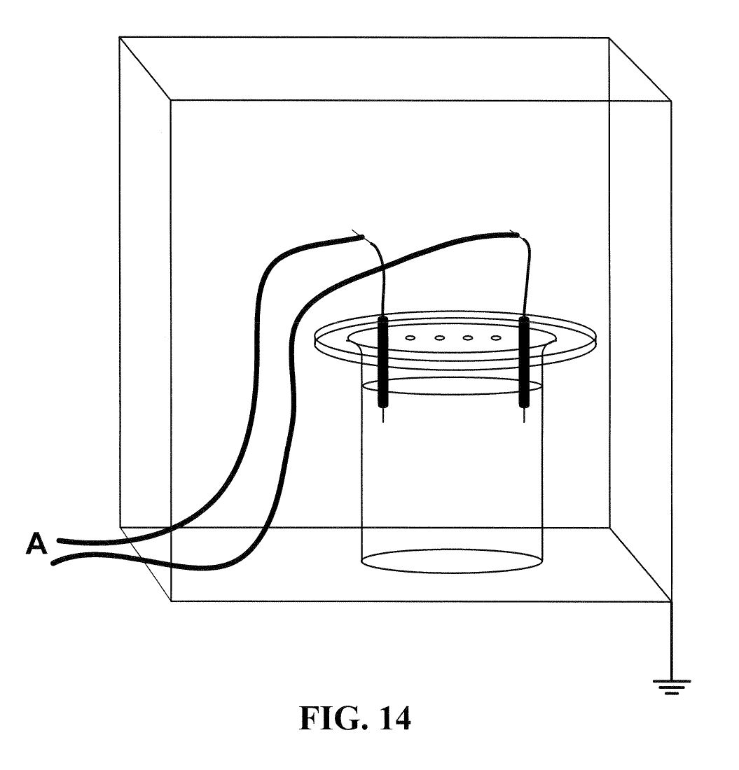

Impedance measurements were taken at 100 Hz, 1 kHz and 10 kHz for both spinulose coated and bare wire (˜250 um in length) using an MCP-BR2822 portable LCR meter, set to a series equivalent circuit, as seen in FIG. 14. 0.45% w/v NaCl was used as an electrolyte in a 150 mL beaker. The electrodes were held at a distance apart using a plate drilled with holes one centimeter apart, center to center, on top of the beaker. The beaker and the electrodes were placed inside an 8″×8″×8″ box made of 0.020″ thick titanium sheet. This box was connected to ground in an attempt to reduce electrical interference. The leads from the LCR meter, illustrated as A in FIG. 14, entered this box and connected to the electrodes. Experiments were done with a 5 cm electrode spacing and 1.5 cm electrode depth.

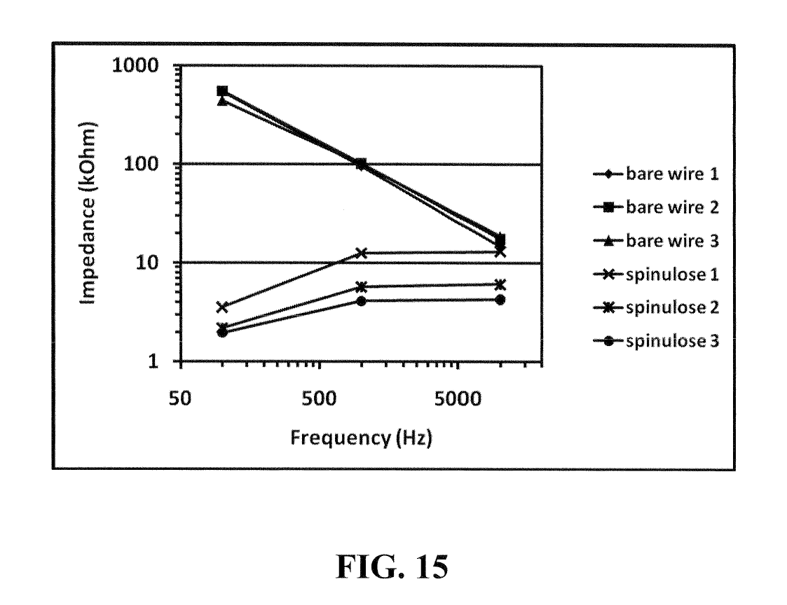

FIG. 15 shows that compared to the bare wire electrodes, the spinulose coated electrodes, with a greater surface area, had a lower impedance over all frequencies measured. FEG-SEM image of a titanium spinulose coating formed from a titanium plasma deposited at an angle of θ_{C}=0° with a θ_{S}=0° on 0.005″ diameter type 302/304 stainless steel wire was used to simulate a miniaturized electrode.

What is claimed is:

1. A spinulose surface comprising randomly spaced irregular nanosized thorn-shaped projections on a rounded metal nanoparticulate adhering to a substrate.

2. The spinulose surface of claim 1 wherein the metal is titanium, zirconium, zirconium/hafnium or mixtures thereof.

3. The spinulose surface of claim 1 wherein the rounded metal nanoparticulate ranges from about 1 to about 10 um in diameter.

4. The spinulose surface of claim 3 wherein projections on the nanoparticulate are about 20% of the nanoparticulate diameter.

5. The spinulose surface of claim 2 further comprising a polymer coating.

6. The spinulose surface of claim 5 wherein the polymer coating is a poly-L-lactic acid (PLLA), poly-(lactic-co-glycolic acid) (PLGA), or a combination of PLLA and PLGA.

7. The spinulose surface of claim 2 further comprising a coating of silver/silver oxide.

8. The spinulose surface of claim 7 further comprising a poly-L-lactic acid (PLLA) coating over the silver/silver oxide.

9. The spinulose surface of claim 2 further comprising an attached osteoblast, fibroblast or endothelial cell.

10. The spinulose surface of claim 9 wherein the fibroblast cell is a human periodontal ligament fibroblast cell or a human gingival cell.

11. The spinulose surface of claim 9 wherein attached cell density is up to at least about 5×10³ cells/cm².

12. The spinulose surface of claim 1 which comprises at least a portion of a medical device.

13. The spinulose surface of claim 12 wherein the medical device is a stent, guidewire, catheter, or electrode.

14. The spinulose surface of claim 13 wherein the electrode is a miniaturized electrode coated with spinulose titanium.

15. The spinulose surface of claim 1 wherein the substrate is substrate is a metal, metal alloy, polymer or ceramic, silicone, poly(methylmethacrylate) (PMMA), polyurethane (PU), polyvinyl chloride (PVC), polyethylene terephthalate (PET), polyethylene terephthalate glycol (PETG), polyetheretherketone (PEEK), ultra high molecular weight polyethylene (UHMWPE), or polypropylene (PP), silicon, glass, carbon, salt, titanium, nitinol, CoCrMo or stainless steel.
